(19) United States
(12) Patent Application Publication
(10) Pub. No.: US 2018/0271358 A1
(43) Pub. Date: Sep. 27, 2018

(54) NAVIGATING AN IMAGING INSTRUMENT IN A BRANCHED STRUCTURE

(71) Applicants: Samaneh Saghatchi, Zanjan (IR); Javad Hasani bidgoli, Tehran (IR); Mohammad Jalal Sadeghi, Tehran (IR); Alireza Ahmadian, Tehran (IR); Farzam Farahmand, Tehran (IR); Saeed Sarkar, Tehran (IR)

(72) Inventors: Samaneh Saghatchi, Zanjan (IR); Javad Hasani bidgoli, Tehran (IR); Mohammad Jalal Sadeghi, Tehran (IR); Alireza Ahmadian, Tehran (IR); Farzam Farahmand, Tehran (IR); Saeed Sarkar, Tehran (IR)

(73) Assignee: Parseh Intelligent Surgical System, Tehran (IR)

(21) Appl. No.: 15/986,724

(22) Filed: May 22, 2018

Related U.S. Application Data
(60) Provisional application No. 62/509,769, filed on May 23, 2017.

(51) Int. Cl. A61B 1/267; A61B 5/06; A61B 6/03; G06K 9/00; G06T 19/00; G06T 7/73

(52) U.S. Cl. CPC A61B 1/2676; A61B 5/064; A61B 6/03; G06T 2207/30061; G06T 19/003; G06T 7/75; G06K 9/00214

(57) ABSTRACT

Disclosed herein is a method for navigating an imaging instrument in a branched structure. The branched structure includes a plurality of landmarks. The method includes selecting a target in the branched structure, selecting a first landmark from the plurality of landmarks, acquiring a virtual model of the branched structure, extracting a first virtual image of the first landmark from the virtual model, acquiring a first live image of the first landmark by the imaging instrument, and registering the first live image with the first virtual image. The first landmark is associated with the target.

### CROSS-REFERENCE TO RELATED APPLICATION

This application claims the benefit of priority from pending U.S. Provisional Patent Application Ser. No. 62/509,769, filed on May 23, 2017, and entitled “OPTIMAL LANDMARK CONFIGURATION DETECTOR FOR BRONCHOSCOPY NAVIGATION,” which is incorporated herein by reference in its entirety.

### TECHNICAL FIELD

The present disclosure generally relates to imaging, and particularly, to image-guided navigation.

### BACKGROUND

Image-guided navigation is an important issue in studying branched structures. As an example, electromagnetic navigation bronchoscopy (ENB) systems are used in imaging pulmonary airways via bronchoscopy, which is a diagnosis technique for lung related disabilities. In this technique, a patient's airways are assessed with a bronchoscope and samples are collected. For example, in case of a lung cancer, a bronchoscope may be navigated utilizing an ENB system inside the pulmonary airways to reach a tumor location. Computed tomography (CT) images may be used as a road map to find the tumor. By utilizing the navigation system and the road map, a suitable location for sampling may be obtained.

A number of landmarks, called fiducials, may be collected to register position data captured by an electromagnetic tracker and the structure of the patient's airways obtained from CT images. In current navigation systems, several fiducials spread all over the patient's airway tree may be selected as landmarks. Due to the large number of landmarks, the navigation process may take considerable time and may increase the probability of inaccuracies related to human errors.

There is, therefore, a need for a navigation method that requires a reduced number of landmarks for imaging branched structures. There is also a need for a navigation method that selects appropriate landmarks for imaging a specific target in a branched structure in a more efficient manner.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general embodiment, the present disclosure describes a method for navigating an imaging instrument in a branched structure. The branched structure may include a plurality of landmarks. The method includes selecting a target in the branched structure, selecting a first landmark from the plurality of landmarks, acquiring a virtual model of the branched structure, extracting a first virtual image of the first landmark from the virtual model, acquiring a first live image of the first landmark by the imaging instrument, and registering the first live image with the first virtual image. The first landmark may be associated with the target.

The above general embodiment may include one or more of the following features. In an exemplary embodiment, the method may further include selecting a second landmark from the plurality of landmarks after acquiring the first live image, extracting a second virtual image of the second landmark from the virtual model, acquiring a second live image of the second landmark by the imaging instrument, replacing the first landmark with the second landmark before registering the first live image with the first virtual image, replacing the first virtual image with the second virtual image before registering the first live image with the first virtual image; and replacing the first live image with the second live image before registering the first live image with the first virtual image. The second landmark may be associated with the target.

In an embodiment, registering the first live image with the first virtual image may include calculating a registration transform by using a live position of the first landmark, a virtual position of the first landmark, and camera specifications of the imaging instrument. The live position of the first landmark may be acquired from the first live image. The virtual position of the first landmark may be acquired from the first virtual image.

In an exemplary embodiment, the method may further include selecting a landmarks subset from the plurality of landmarks in a neighborhood of the target, extracting a first plurality of virtual images from the virtual model by extracting an image of each of the landmarks in the landmarks subset from the virtual model, acquiring a plurality of live images via imaging each of the landmarks in the landmarks subset by the imaging instrument, and registering the plurality of live images with the plurality of virtual images. The landmarks subset may be associated with the first landmark.

In an exemplary embodiment, registering the plurality of live images with the first plurality of virtual images may include acquiring a plurality of live positions by extracting positions of at least three landmarks in the landmarks subset from the plurality of live images, acquiring a second plurality of virtual positions by extracting positions of the three landmarks in the landmarks subset from the first plurality of virtual images, and calculating a registration transform by using the plurality of live positions and the second plurality of virtual positions.

In an embodiment, navigating the imaging instrument in the branched structure may include navigating a bronchoscope in a bronchial tree. The bronchoscope may be associated with a tracking instrument. The bronchial tree associated with a main carina, a right lung, and a left lung. The right lung may include a right main bronchus, an intermediate bronchus, a right upper lobar bronchus, a right middle lobar bronchus, and a right lower lobar bronchus. The left lung may include a left main bronchus, a left upper lobar bronchus, a left lingular bronchus, and a left lower lobar bronchus.

In an exemplary embodiment, responsive to determining that the target being located in a right upper lobe of the right lung, selecting the landmarks subset may include selecting each landmark in the landmarks subset from inside a first region. The first region may include the right upper lobe and a middle lobe of the right lung.

In an embodiment, responsive to determining that the target being located in a middle lobe of the right lung, selecting the landmarks subset may include selecting each landmark in the landmarks subset from inside a second region. The second region may include the right upper lobe and the middle lobe.

In an exemplary embodiment, responsive to determining that the target being located in a right lower lobe of the right lung, selecting the landmarks subset may include selecting each landmark in the landmarks subset from inside a third region. The third region may include the right lower lobe.

In an embodiment, responsive to determining that the target being located in a left upper lobe of the left lung, selecting the landmarks subset may include selecting each landmark in the landmarks subset from inside a fourth region. The fourth region may include the left upper lobe.

In an exemplary embodiment, responsive to determining that the target being located in a left lower lobe of the left lung, selecting the landmarks subset may include selecting each landmark in the landmarks subset from inside a fifth region. The fifth region may include the left lower lobe.

In an exemplary embodiment, responsive to determining that the target being located in the right upper lobe, selecting the landmarks subset may include selecting a first bifurcation of the main carina into the left main bronchus and the right main bronchus, a second bifurcation of the right upper lobar bronchus into an apical segment and a posterior segment, a third bifurcation of the right upper lobar bronchus into the apical segment and a right anterior segment, and a fourth bifurcation of the right upper lobar bronchus into the posterior segment and the right anterior segment.

In an embodiment, responsive to determining that the target being located in the middle lobe, selecting the landmarks subset may include selecting the first bifurcation, a fifth bifurcation of the right middle lobar bronchus into a lateral segment and a medial segment, a sixth bifurcation of the right lower lobar bronchus into a right anterior basal segment and a right medial basal segment, and a seventh bifurcation of the right lower lobar bronchus into a right lateral basal segment and a right posterior basal segment.

In an exemplary embodiment, responsive to determining that the target being located in the right lower lobe, selecting the landmarks subset may include selecting the fifth bifurcation, the seventh bifurcation, an eighth bifurcation of the intermediate bronchus into the right middle lobar bronchus and the right lower lobar bronchus, and a ninth bifurcation of the right lower lobar bronchus into the right anterior basal segment and the right lateral basal segment.

In an exemplary embodiment, responsive to determining that the target being located in the left upper lobe, selecting the landmarks subset may include selecting the first bifurcation, a tenth bifurcation of the left main bronchus into the left upper lobar bronchus and the left lower lobar bronchus, an eleventh bifurcation of the left upper lobar bronchus into an apicoposterior segment and a left anterior segment, and a twelfth bifurcation of the left lingular bronchus into a superior lingular segment and an inferior lingular segment.

In an embodiment, responsive to determining that the target being located in the left lower lobe, selecting the landmarks subset may include selecting the tenth bifurcation, a thirteenth bifurcation of the left lower lobar bronchus into a left medial basal segment and a left posterior basal segment, a fourteenth bifurcation of the left lower lobar bronchus into a left anterior basal segment and the left medial basal segment, and a fifteenth bifurcation of the left lower lobar bronchus into a left lateral basal segment and the left posterior basal segment.

In an exemplary embodiment, acquiring the plurality of live positions may include moving a tip of the of the bronchoscope to a position of a landmark in the landmarks subset by using a corresponding live image of the plurality of live images, and extracting the tip position by using the tracking instrument.

In an embodiment, acquiring the virtual model may include reconstructing a three-dimensional model of the bronchial tree from a plurality of computed tomography (CT) images of the bronchial tree.

Other systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of an exemplary embodiment of a method for navigating an imaging instrument in a branched structure, consistent with exemplary embodiments of the present disclosure.

FIG. 1B shows a flowchart of an exemplary embodiment the method for navigating an imaging instrument in a branched structure with additional steps, consistent with exemplary embodiments of the present disclosure.

FIG. 1C shows a flowchart of an exemplary embodiment of the method for navigating an imaging instrument in a branched structure with additional steps to update a registration transform, consistent with exemplary embodiments of the present disclosure.

FIG. 1D shows a flowchart of an exemplary embodiment of selecting a landmarks subset from a plurality of landmarks, consistent with exemplary embodiments of the present disclosure.

FIG. 1E shows a flowchart of an exemplary embodiment of registering a plurality of live images with a plurality of virtual images, consistent with exemplary embodiments of the present disclosure.

FIG. 2 shows an exemplary branched structure that is being imaged by an exemplary imaging instrument, consistent with exemplary embodiments of the present disclosure.

FIG. 3 shows an exemplary embodiment of a bronchial tree, consistent with exemplary embodiments of the present disclosure.

FIG. 4A shows an exemplary virtual model of a branched structure, consistent with exemplary embodiments of the present disclosure.

FIG. 4B shows an example embodiment of a virtual image, consistent with exemplary embodiments of the present disclosure.

FIG. 4C shows an exemplary embodiment of a live image, consistent with exemplary embodiments of the present disclosure.

FIG. 5 shows a schematic representation for extracting a plurality of virtual images from a virtual model, consistent with exemplary embodiments of the present disclosure.

FIG. 6 shows a schematic representation for registering a plurality of live images with a plurality of virtual images, consistent with exemplary embodiments of the present disclosure.

FIG. 7 shows an exemplary embodiment of a live image, consistent with exemplary embodiments of the present disclosure.

FIG. 8 shows a plurality of images of a patient's airways, consistent with exemplary embodiments of the present disclosure.

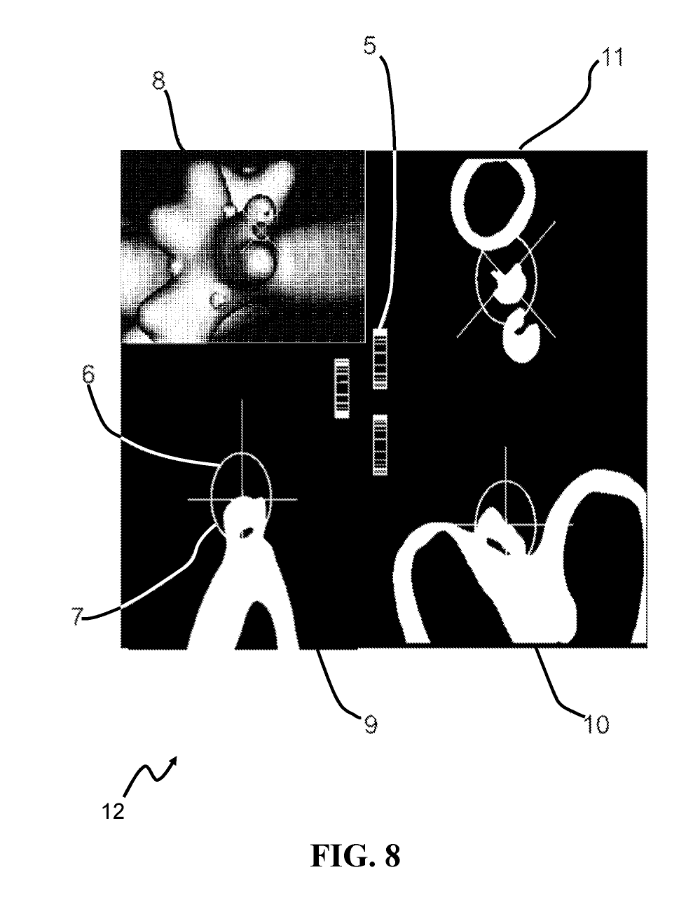

FIG. 9 shows a three-dimensional map of a patient's tracheobronchial tree, consistent with exemplary embodiments of the present disclosure.

FIG. 10 shows a plurality of images of a patient's airways, consistent with exemplary embodiments of the present disclosure.

FIG. 11 shows a side-by-side view of a live image and a virtual image, consistent with exemplary embodiments of the present disclosure.

FIG. 12 shows an overlaid view of a live image and a virtual image, consistent with exemplary embodiments of the present disclosure.

FIG. 13 shows an overlaid view of a live image and a virtual image after a tip of a bronchoscope has reached a location of a selected landmark, consistent with exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed a method for navigating an imaging instrument in a branched structure. The method includes selecting a target that is to be imaged, selecting a group of landmarks in the branches, and registering a pair of images of each landmark. A first image is captured by an imaging instrument (i.e., the live image), and a second image is extracted from a virtual model (e.g., a three-dimensional image). The second image may show a landmark in a cross-section virtual image. If the live image of the landmark is not satisfactory, the landmark may be replaced with a new landmark and the registration procedure may be repeated. The registration procedure is updated by selecting a new landmark from a group of landmarks, until the last landmark is processed. The group of landmarks are selected according to an approximate location of the target.

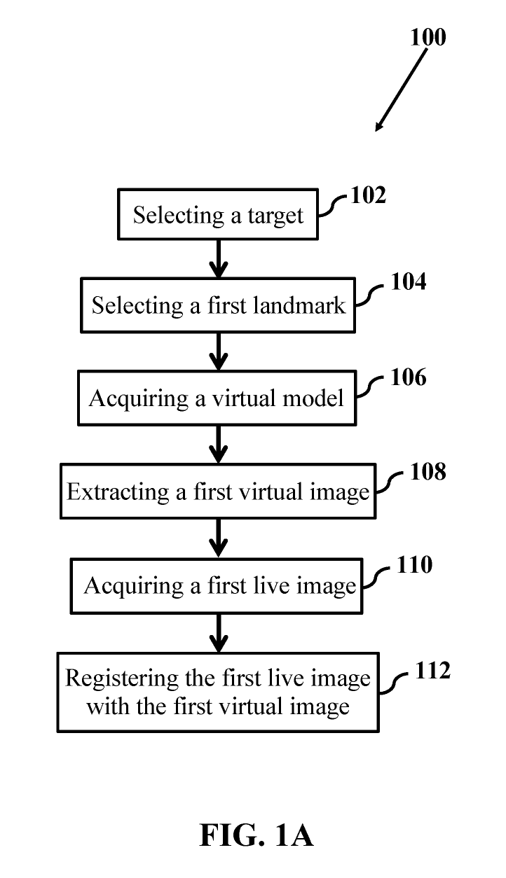

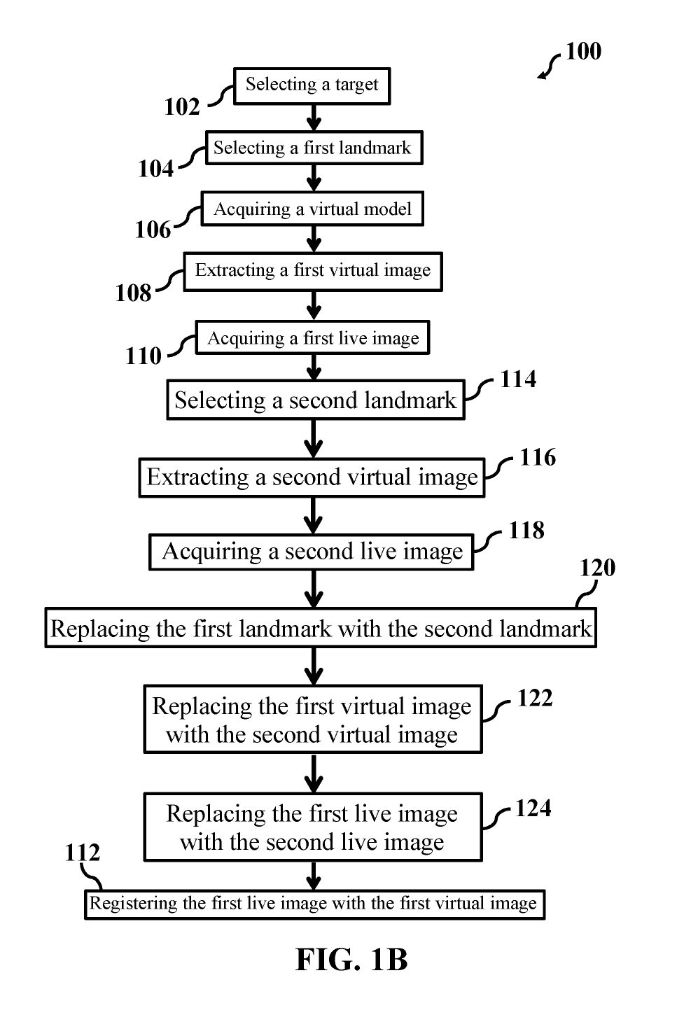

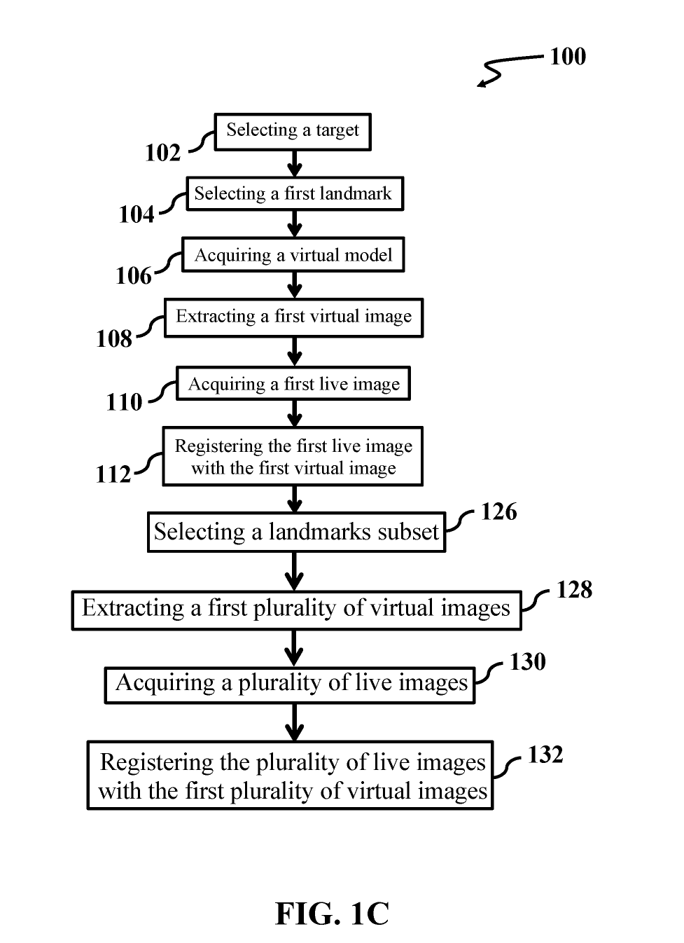

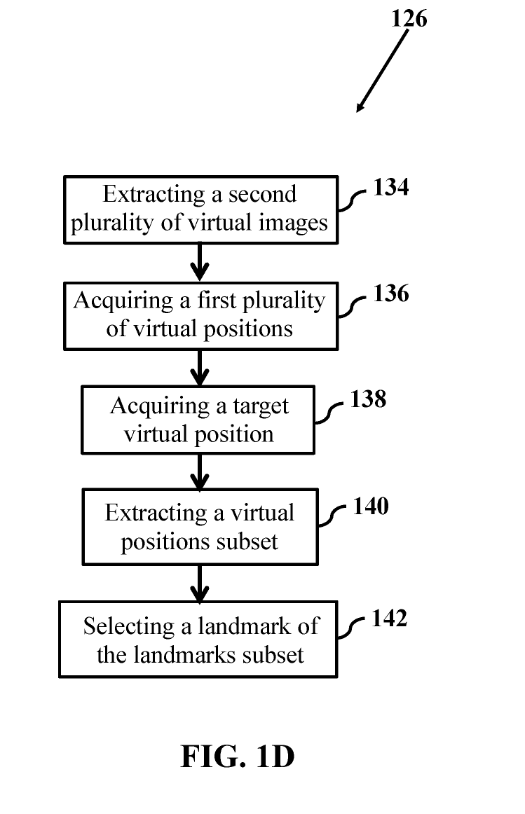

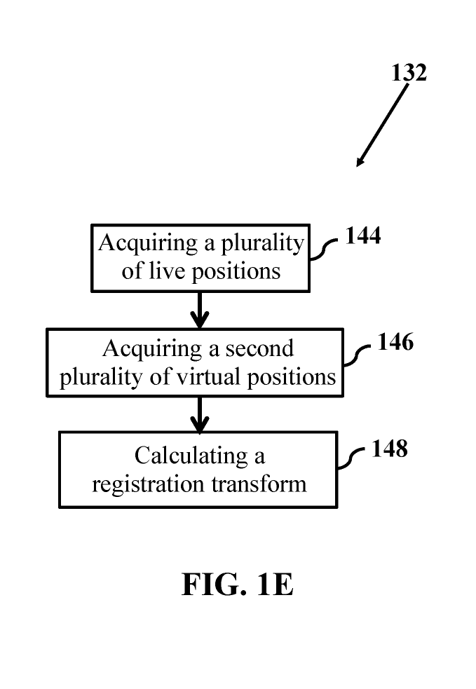

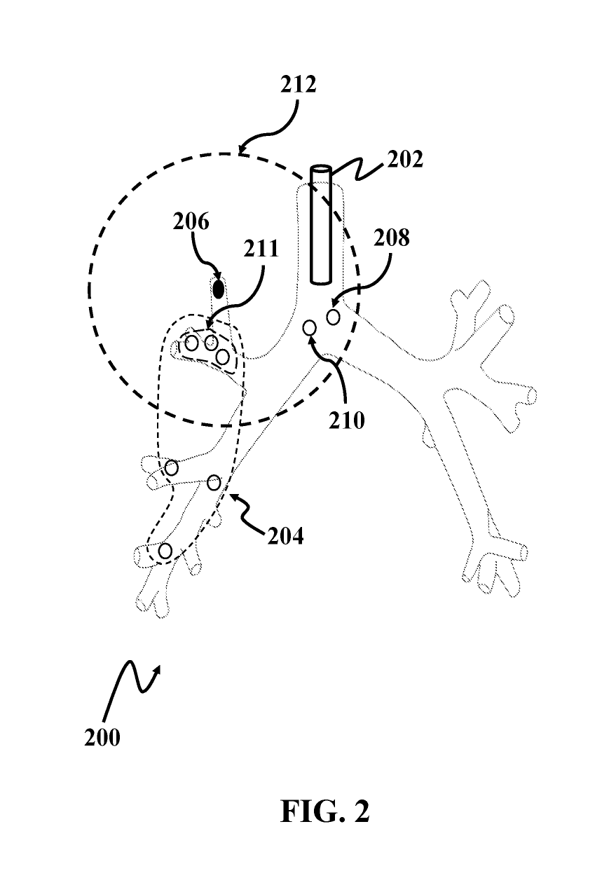

FIG. 1A shows a flowchart of an exemplary embodiment of a method 100 for navigating an imaging instrument in a branched structure, consistent with exemplary embodiments of the present disclosure. FIG. 2 shows an exemplary branched structure 200 that is being imaged by an exemplary imaging instrument 202, consistent with exemplary embodiments of the present disclosure. branched structure 200 may include a plurality of landmarks 204. Referring to FIG. 1A and FIG. 2, in some examples, method 100 may include selecting a target 206 in branched structure 200 (step 102), selecting a first landmark 208 from plurality of landmarks 204 (step 104), acquiring a virtual model of branched structure 200 (step 106), extracting a first virtual image of first landmark 208 from the virtual model (step 108), acquiring a first live image of first landmark 208 by imaging instrument 202 (step 110), and registering the first live image with the first virtual image (step 112). In some exemplary implementations, first landmark 208 may be associated with target 206.

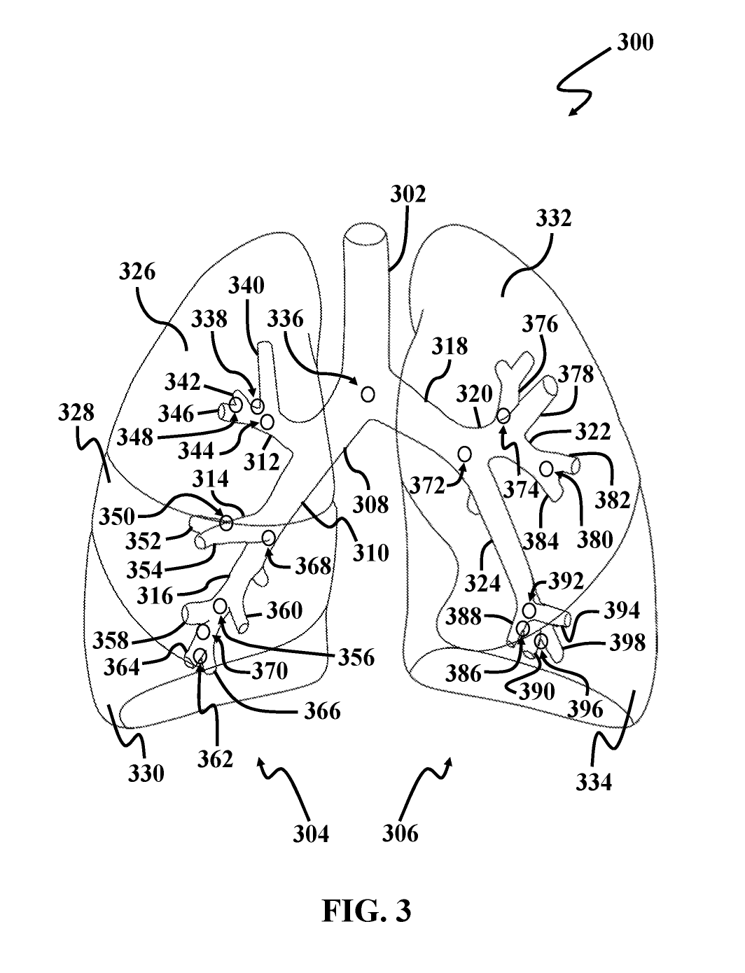

According to an exemplary embodiment, navigating imaging instrument 202 in branched structure 200 may include navigating a bronchoscope in a bronchial tree. The bronchoscope may be associated with a tracking instrument. FIG. 3 shows an exemplary embodiment of bronchial tree 300, consistent with exemplary embodiments of the present disclosure. The bronchial tree may be associated with a main carina 302, a right lung 304, and a left lung 306. In an embodiment, right lung 304 may include a right main bronchus 308, an intermediate bronchus 310, a right upper lobar bronchus 312, a right middle lobar bronchus 314, and a right lower lobar bronchus 316. In an embodiment, left lung 306 may include a left main bronchus 318, a left upper lobar bronchus 320, a left lingular bronchus 322, and a left lower lobar bronchus 324.

Referring again to FIGS. 1-3, step 106 may include acquiring a virtual model of branched structure 200. In an exemplary embodiment, acquiring virtual model 402 may include reconstructing a three-dimensional model of bronchial tree 300 from a plurality of computed tomography (CT) images of bronchial tree 300.

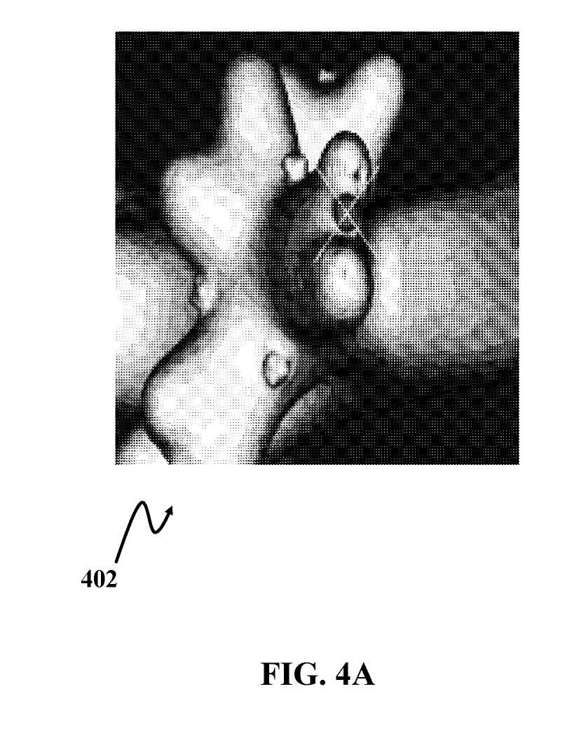

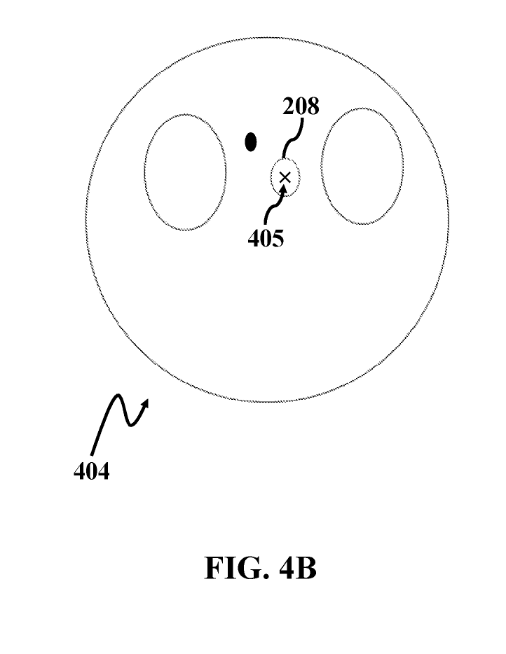

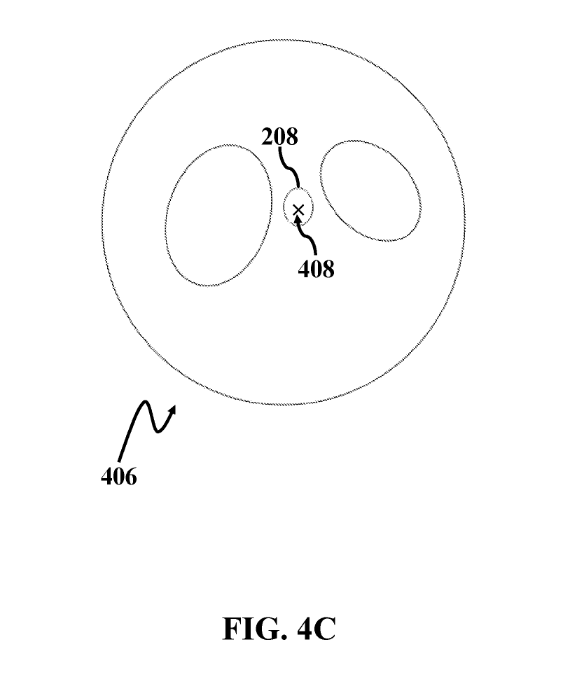

For the purpose of clarity, FIG. 4A shows an exemplary virtual model 402 of branched structure 200, consistent with exemplary embodiments of the present disclosure. In some exemplary embodiments, virtual model 402 may include a given three-dimensional image of branched structure 200. FIG. 4B shows an example embodiment of first virtual image 404, consistent with exemplary embodiments of the present disclosure. In some embodiments, first virtual image 404 may include a two-dimensional cross-section of virtual model 402 that includes an image of first landmark 208. FIG. 4C shows an exemplary embodiment of first live image 406 that may be captured by imaging instrument 202 of first landmark 208 during the imaging process.

Referring again to FIGS. 1 and 2, if the live and virtual images of first landmark 208 are not satisfactory, first landmark 208 may be replaced with a second landmark 210, and additional steps may be added to method 100 to acquire virtual and live images for second landmark 210. FIG. 1B shows a flowchart of an exemplary embodiment of method 100 with additional steps. In an exemplary embodiment, method 100 may further include selecting second landmark 210 from plurality of landmarks 204 (step 114) after acquiring first live image 406 (step 110), extracting a second virtual image of second landmark 210 from virtual model 402 (step 116), acquiring a second live image of second landmark 210 by imaging instrument 202 (step 118), replacing first landmark 208 with second landmark 210 (step 120) before registering first live image 406 with first virtual image 404 (step 112), replacing first virtual image 404 with the second virtual image (step 122) before registering first live image 406 with first virtual image 404 (step 112), and replacing first live image 406 with the second live image (step 124) before registering first live image 406 with first virtual image 404 (step 112). The second landmark may be associated with target 206. In an exemplary embodiment, the second virtual image may include a two-dimensional cross-section of virtual model 402 that includes an image of the second landmark. In another embodiment, the second live image may be taken by imaging instrument 202 of the second landmark during the imaging process.

Referring again to FIGS. 1 and 4, step 112 may include registering first live image 406 with first virtual image 404. In an exemplary embodiment, registering first live image 406 with first virtual image 404 may include calculating a registration transform by using a virtual position 305 of first landmark 208, a live position 308 of first landmark 208, and camera specifications of imaging instrument 202. live position 308 may be acquired from first live image 406, and virtual position 305 may be acquired from first virtual image 404.

Referring again to FIGS. 1, 2, and 4, in an exemplary embodiment, the registration transform may be updated by utilizing one or more of plurality of landmarks 204, to achieve a higher precision in the registration process. FIG. 1C shows a flowchart of an exemplary embodiment of method 100 with additional steps to update the registration transform, consistent with exemplary embodiments of the present disclosure. In an embodiment, method 100 may further include selecting a landmarks subset 211 from plurality of landmarks 204 in a neighborhood 212 of target 206 (step 126), extracting a first plurality of virtual images from virtual model 402 by extracting an image of each of the landmarks in landmarks subset 211 from virtual model 402 (step 128), acquiring a plurality of live images via imaging each of the landmarks in landmarks subset 211 by imaging instrument 202 (step 130), and registering the plurality of live images with the first plurality of virtual images (step 132).

Step 126 may include selecting landmarks subset 211. In an embodiment, landmarks subset 211 may be associated with first landmark 208. If first landmark 208 is replaced with second landmark 210, landmarks subset 211 may also be accordingly modified, to be consistent with second landmark 210. FIG. 1D shows a flowchart of an exemplary embodiment of step 126, consistent with exemplary embodiments of the present disclosure. In an exemplary embodiment, selecting landmarks subset 211 may include extracting a second plurality of virtual images from virtual model 402 by extracting an image of each of plurality of landmarks 204 in neighborhood 212 of target 206 from virtual model 402 (step 134), acquiring a first plurality of virtual positions by extracting positions of each of plurality of landmarks 204 from the second plurality of virtual images (step 136), acquiring a target virtual position by extracting a position of the target from a target virtual image (step 138), extracting a virtual positions subset from the first plurality of virtual positions such that a geometric center of the virtual positions subset coincides with the target virtual position (step 140), and selecting a landmark of the landmarks subset from the plurality of landmarks (step 142). The position of the landmark in the virtual image of the landmark may be included in the virtual positions subset. In an embodiment, the target virtual image may be extracted from the virtual model and may include an image of the target.

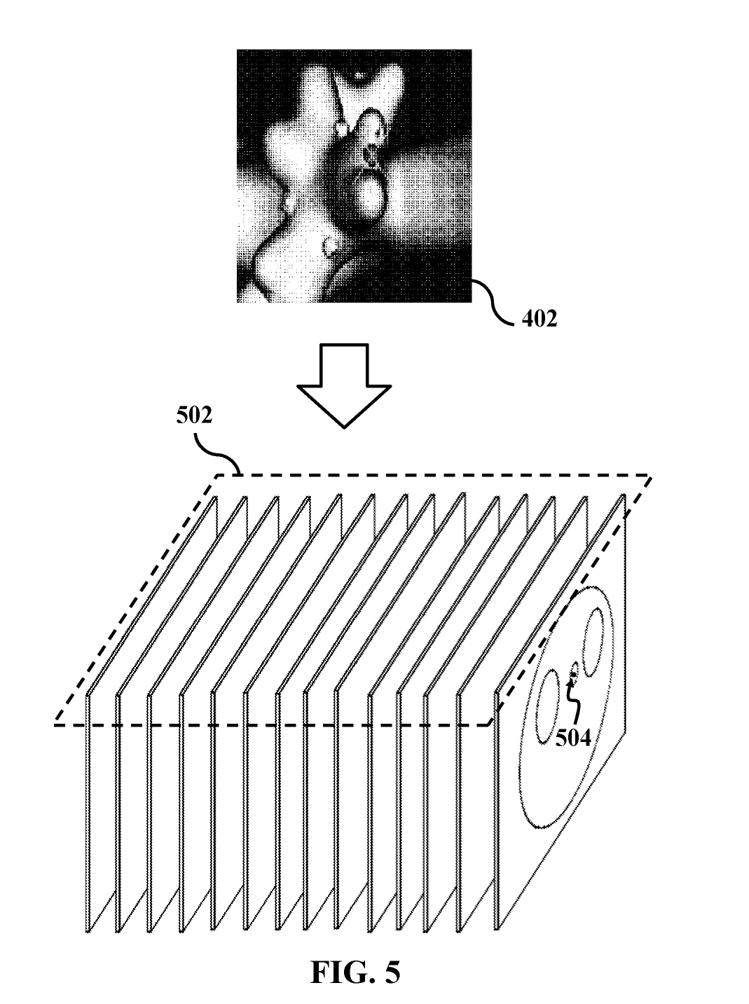

Step 134 may include extracting the second plurality of virtual images from virtual model 402. For further clarification, FIG. 5 shows a schematic representation for extracting a second plurality of virtual images 502 from virtual model 402. In an exemplary embodiment, each of the second plurality of virtual images may include a two-dimensional cross-section of virtual model 402. Each of second plurality of virtual images 502 may include an image of a landmark of plurality of landmarks 204. Step 136 may include acquiring the first plurality of virtual positions from second plurality of virtual images 502. For a landmark of plurality of landmarks 204, a virtual position 504 of the first plurality of virtual positions may be obtained by extracting a position of the landmark in a corresponding virtual image of second plurality of virtual images 502. The corresponding virtual image may include an image of the landmark.

Referring again to FIGS. 1C and 3, in an exemplary embodiment, responsive to determining that target 206 being located in a right upper lobe 326 of right lung 304, selecting landmarks subset 211 (step 126) may include selecting each landmark in landmarks subset 211 from inside a first region. The first region may include right upper lobe 326 and middle lobe 328. In an embodiment, responsive to determining that target 206 being located in a middle lobe 328 of right lung 304, selecting landmarks subset 211 (step 126) may include selecting each landmark in landmarks subset 211 from inside a second region. The second region may include right upper lobe 326 and middle lobe 328. In an exemplary embodiment, responsive to determining that target 206 being located in a right lower lobe 330 of right lung 304, selecting landmarks subset 211 (step 126) may include selecting each landmark in landmarks subset 211 from inside a third region. The third region may include right lower lobe 330. In an embodiment, responsive to determining that target 206 being located in a left upper lobe 332 of left lung 306, selecting landmarks subset 211 (step 126) may include selecting each landmark in landmarks subset 211 from inside a fourth region. The fourth region may include left upper lobe 332. In an exemplary embodiment, responsive to determining that target 206 being located in a left lower lobe 334 of left lung 306, selecting landmarks subset 211 (step 126) may include selecting each landmark in landmarks subset 211 from inside a fifth region. The fifth region may include left lower lobe 334.

In an embodiment, responsive to determining that target 206 being located in right upper lobe 326, selecting landmarks subset 211 (step 126) may include selecting a first bifurcation 336 of main carina 302 into left main bronchus 318 and right main bronchus 308, selecting a second bifurcation 338 of right upper lobar bronchus 312 into an apical segment 340 and a posterior segment 342, selecting a third bifurcation 344 of right upper lobar bronchus 312 into apical segment 340 and a right anterior segment 346, and selecting a fourth bifurcation 348 of right upper lobar bronchus 312 into posterior segment 342 and right anterior segment 346.

In an exemplary embodiment, responsive to determining that target 206 being located in middle lobe 328, selecting landmarks subset 211 (step 126) may include selecting first bifurcation 336, selecting a fifth bifurcation 350 of right middle lobar bronchus 314 into a lateral segment 352 and a medial segment 354, selecting a sixth bifurcation 356 of right lower lobar bronchus 316 into a right anterior basal segment 358 and a right medial basal segment 360, and selecting a seventh bifurcation 362 of right lower lobar bronchus 316 into a right lateral basal segment 364 and a right posterior basal segment 366.

In an embodiment, responsive to determining that target 206 being located in right lower lobe 330, selecting landmarks subset 211 (step 126) may include selecting fifth bifurcation 350, selecting seventh bifurcation 362, selecting an eighth bifurcation 368 of intermediate bronchus 310 into right middle lobar bronchus 314 and right lower lobar bronchus 316, and selecting a ninth bifurcation 370 of right lower lobar bronchus 316 into right anterior basal segment 358 and right lateral basal segment 364.

In an exemplary embodiment, responsive to determining that target 206 being located in left upper lobe 332, selecting landmarks subset 211 (step 126) may include selecting first bifurcation 336, selecting a tenth bifurcation 372 of left main bronchus 318 into left upper lobar bronchus 320 and left lower lobar bronchus 324, selecting an eleventh bifurcation 374 of left upper lobar bronchus 320 into an apicoposterior segment 376 and a left anterior segment 378, and selecting a twelfth bifurcation 380 of left lingular bronchus 322 into a superior lingular segment 382 and an inferior lingular segment 384.

In an embodiment, responsive to determining that target 206 being located in left lower lobe 334, selecting landmarks subset 211 (step 126) may include selecting tenth bifurcation 372, selecting a thirteenth bifurcation 386 of left lower lobar bronchus 324 into a left medial basal segment 388 and a left posterior basal segment 390, selecting a fourteenth bifurcation 392 of left lower lobar bronchus 324 into a left anterior basal segment 394 and left medial basal segment 388, and selecting a fifteenth bifurcation 396 of left lower lobar bronchus 324 into a left lateral basal segment 398 and left posterior basal segment 390.

Referring again to FIG. 1C, step 132 may include registering the plurality of live images with the first plurality of virtual images. FIG. 1E shows a flowchart of an exemplary embodiment of step 132, consistent with exemplary embodiments of the present disclosure. In an exemplary embodiment, registering the plurality of live images with the first plurality of virtual images may include acquiring a plurality of live positions by extracting positions of at least three landmarks in landmarks subset 211 from the plurality of live images (step 144), acquiring a second plurality of virtual positions by extracting positions of the three landmarks in landmarks subset 211 from the first plurality of virtual images (step 146), and calculating a registration transform by using the plurality of live positions and the second plurality of virtual positions (step 148). In an embodiment, calculating the registration transform may include calculating a transformation matrix T by solving a set of equations. Each equation in the set of equations may be a matrix equation TX=Y, where X is a vector that includes coordinates of a live position of the plurality of live positions (for example, a landmark position in an electromagnetic tracking coordinate system obtained by an electromagnetic tracking sensor), and Y is a vector that includes coordinates of a virtual position of the second plurality of virtual positions (i.e., a landmark position in a virtual model coordinate system, for example a computed tomography image coordinate system). The virtual position may correspond to the live position. In an embodiment, at least three equations may be required to calculate the transformation matrix T. For each equation, a pair of a live position and a corresponding virtual position may be needed. If the number of the plurality of live positions is larger than three (i.e., more than three landmarks are available), the transformation matrix T may be more precisely calculated by adding more equations to the set of equations.

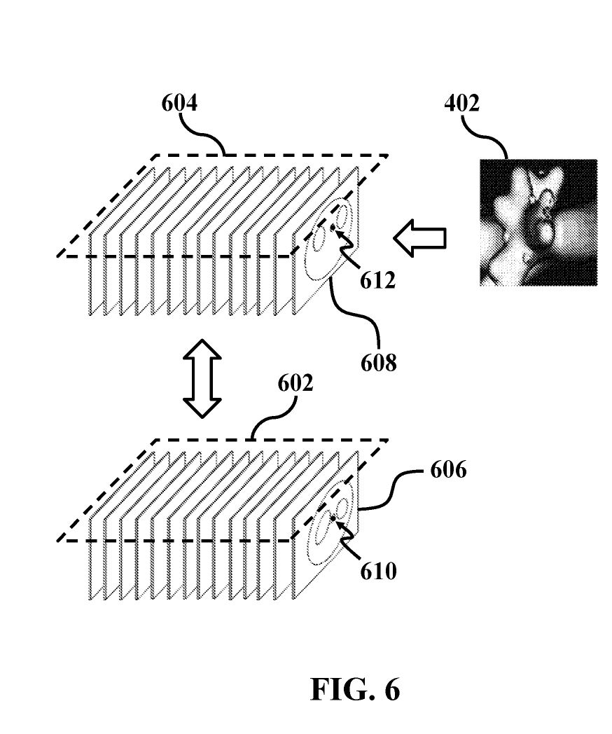

FIG. 6 shows a schematic representation for registering a plurality of live images 602 with a first plurality of virtual images 604 (step 132), consistent with exemplary embodiments of the present disclosure. In an embodiment, each of the plurality of live images may be captured by imaging instrument 202 of a landmark in landmarks subset 211 during the imaging process. Each of first plurality of virtual images 604 may include a two- dimensional cross-section of virtual model 402 that includes an image of a landmark in landmarks subset 211. In an exemplary embodiment, for each landmark in landmarks subset 211, there may exist a pair of images, including a third live image 606 of plurality of live images 602, and a third virtual image 608 of first plurality of virtual images 604. Both third live image 606 and third virtual image 608 may include an image of a same landmark in landmarks subset 211. In an exemplary embodiment, registering plurality of live images 602 with first plurality of virtual images 604 may include performing a registration process between third live image 606 and third virtual image 608. The registration process may be similarly repeated for each landmark in landmarks subset 211 to enhance the precision of the registration process, until all landmarks in landmarks subset 211 are processed.

Referring to FIGS. 1E and 6, step 144 may include acquiring the plurality of live positions. In an exemplary embodiment, for a landmark in landmarks subset 211, a live position 610 of the plurality of live positions may be obtained by extracting a position of the landmark in a corresponding live image of plurality of live images 602. The corresponding live image may include an image of the landmark. Step 146 may include acquiring the second plurality of virtual positions. In an exemplary embodiment, for a landmark in landmarks subset 211, a virtual position 612 of the second plurality of virtual positions may be obtained by extracting a position of the landmark in a corresponding virtual image of first plurality of virtual images 604. The corresponding virtual image may include an image of the landmark.

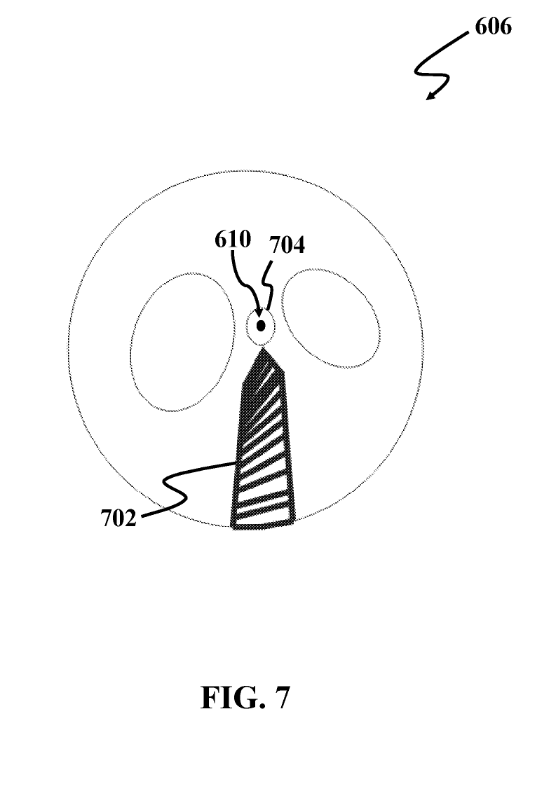

FIG. 7 shows an exemplary embodiment of third live image 606, consistent with exemplary embodiments of the present disclosure. In an exemplary embodiment, acquiring the plurality of live positions (step 144) may include moving a tip 702 of the of the bronchoscope to a live position 610 of a landmark 704 in landmarks subset 211, and extracting the tip 702 position by using the tracking instrument. In an embodiment, tip 702 may be moved to a live position 610 by using third live image 606 of the plurality of live images.

### EXAMPLE

In this example, a software module is presented for navigating an exemplary bronchoscopy system by using an embodiment of method 100.

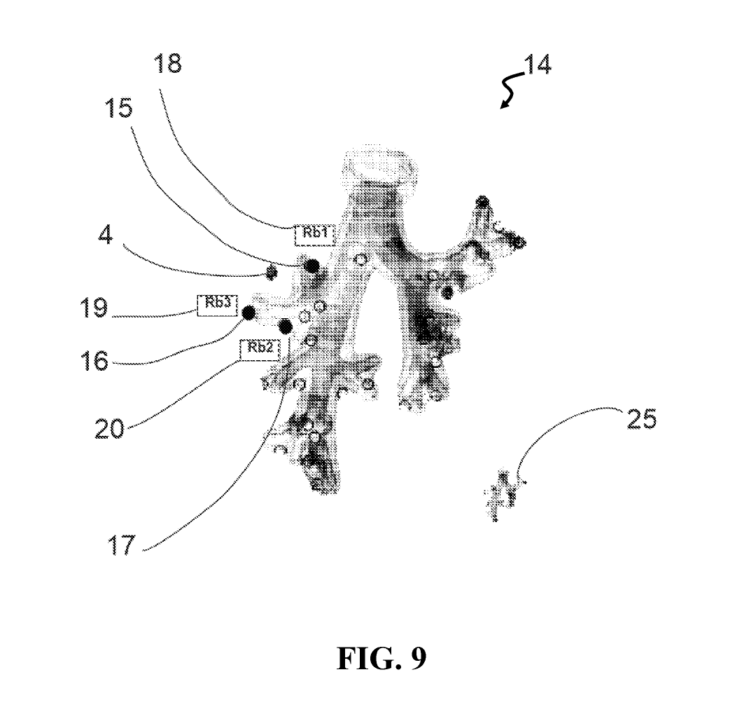

FIG. 9 shows a plurality of images 12 of a patient airways. A first two-dimensional slice 9, a second two-dimensional slice 10, and a third two-dimensional slice 11 include images of the patient airways that are extracted from a three-dimensional virtual model 8. Three-dimensional virtual model 8 is taken using a computed tomography (CT) imaging system. Three-dimensional virtual model 8 is acquired by segmentation of the patient's volume data from the trachea till the fourth-generation bronchial tree.

A cross-shaped control 6 identifies a location of a lung nodule in the patient's airways. Control 6 may be dragged to select the nodule area in any of first two-dimensional slice 9, second two-dimensional slice 10, and third two-dimensional slice 11. A control 7 changes the radius of a selected area. When a user clicks on control 6 or adjusts the selected area by control 7, the changes appear on the plurality of images 12, and the selected area is highlighted. A slide control 5 may be used to change one of first two-dimensional slice 9, second two-dimensional slice 10, and third two-dimensional slice 11.

FIG. 9 shows a three-dimensional map 14 of the patient's tracheobronchial tree. Map 14 is obtained by a segmentation of the patient's airways in three-dimensional virtual model 8. Based on a position of a specific target, a recommended landmarks subset, including landmark 15, landmark 16, and landmark 17 and their anatomical tags, including tag 18, tag 19, and tag 20 are selected and displayed by the module. The corresponding airways that lead to the target area highlighted in map 14 to facilitate recognizing the position of each landmark in the recommended landmarks subset. Therefore, the bronchoscope may be more easily moved to each landmark.

Each landmark in the recommended landmarks subset is displayed by a sphere and corresponding tags, including tag 18, tag 19 and tag 20. Each of the tags includes an anatomical label based on the nearest division's name. A control 25 symbolizes the user's view of map 14. The user's view may be changed by rotating it control 25.

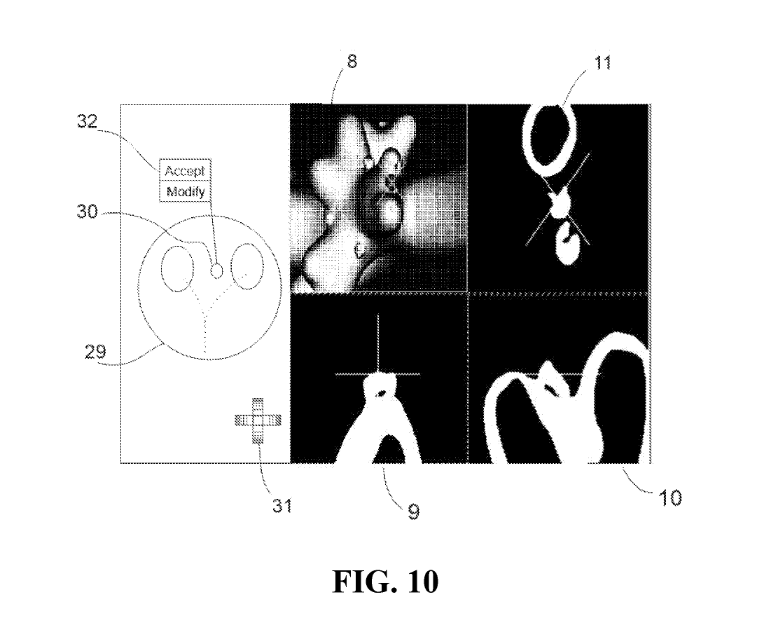

FIG. 10 includes the plurality of images of the patient airways. First two-dimensional slice 9, second two-dimensional slice 10, and third two-dimensional slice 11 are orthogonal two-dimensional slices of three-dimensional virtual model 8, according to a selected landmark 30. A virtual image 29 is a virtual bronchoscopy view of selected landmark 30 that visualizes the lumen and wall of the airway which includes selected landmark 30.

A control 31 may be used for adjusting a view of virtual image 29. The adjusted view may be saved as a golden virtual image to be used for navigating the bronchoscope to the landmark position. If selected landmark 30 is acceptable after acquiring the golden virtual image, selected landmark 30 may be accepted by choosing an accept command in a pop-up window 32. Otherwise, selected landmark 30 may be replaced by another landmark by choosing a modify command in pop-up window 32.

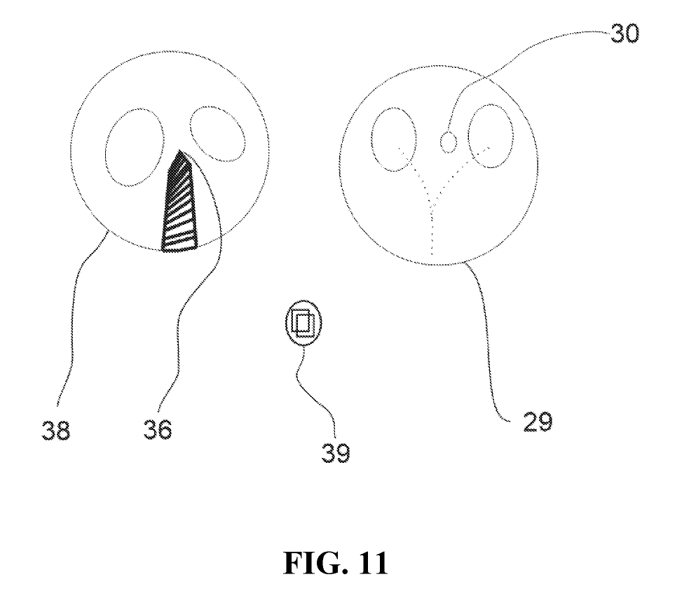

FIG. 11 shows a side-by-side view of a live image 38 and virtual image 29. Live image 34 is captured by a bronchoscope camera, and includes tracking data recorded by an electromagnetic tracking sensor inside a working channel of the bronchoscope. The bronchoscope may be moved to an approximate location of selected landmark 30 by using the tracking data of live image 38. When the bronchoscope approaches the location of selected landmark 30, a tip 36 of the electromagnetic tracking sensor inside the working channel of the bronchoscope may be seen in live image 38. At this point, live image 34 may be compared with virtual image 29 to obtain the position of selected landmark 37 position in live image 38. A control 39 may be used to change the side-by-side view of live image 38 and virtual image 29 to an overlaid view.

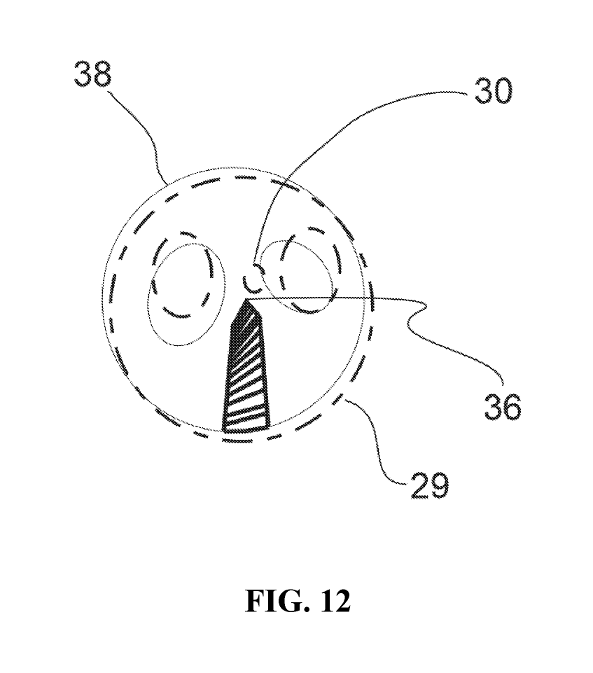

FIG. 12 shows an overlaid view of live image 38 and virtual image 29. The overlaid view may provide real-time visual feedback of the bronchoscopy navigation to enhance steering the tip 36 to the location of selected landmark 30.

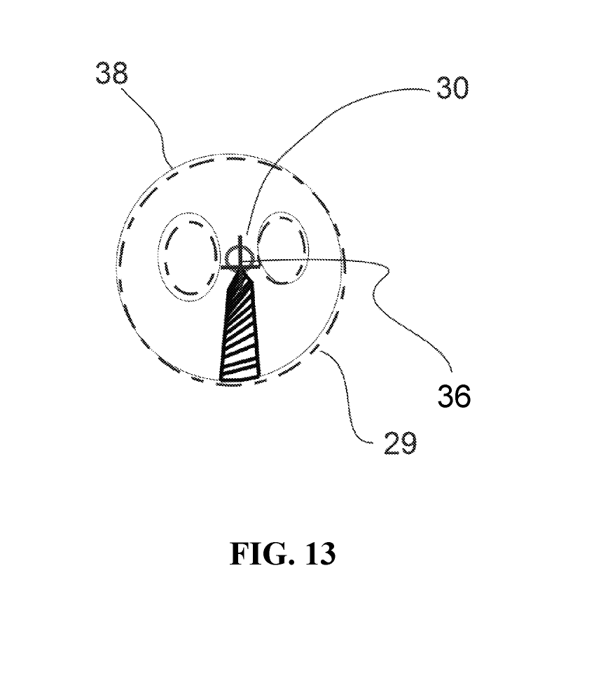

FIG. 13 shows an overlaid view of live image 38 and virtual image 29 after tip 36 has reached the location of selected landmark 30. Referring again to FIG. 10, the position of selected landmark 30 may be confirmed by choosing the accept command in pop up window 32. This process may be repeated for all landmarks in the recommended landmarks subset to collect the positions of all recommended landmarks in the live images the virtual images. A registration transform may be calculated for registering the live images with the virtual images by using the positions of landmarks in the recommended landmarks subset.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it may be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for navigating a bronchoscope associated with a tracking instrument in a bronchial tree, the bronchial tree associated with a main carina, a right lung, and a left lung, the right lung comprising a right main bronchus, an intermediate bronchus, a right upper lobar bronchus, a right middle lobar bronchus, and a right lower lobar bronchus, the left lung comprising a left main bronchus, a left upper lobar bronchus, a left lingular bronchus, and a left lower lobar bronchus, the bronchial tree comprising a plurality of landmarks, the method comprising:
selecting a target in the bronchial tree;
selecting a first landmark from the plurality of landmarks, the first landmark associated with the target;
acquiring a virtual model by reconstructing a three-dimensional model of the bronchial tree from a plurality of computed tomography (CT) images of the bronchial tree;
extracting a first virtual image of the first landmark from the virtual model;
acquiring a first live image of the first landmark by the bronchoscope;
selecting a second landmark from the plurality of landmarks, the second landmark associated with the target;
extracting a second virtual image of the second landmark from the virtual model;
acquiring a second live image of the second landmark by the bronchoscope;
calculating a registration transform by using a live position of the second landmark acquired from the second live image, a virtual position of the second landmark acquired from the second virtual image and camera specifications of the bronchoscope;
selecting a landmarks subset from the plurality of landmarks, comprising:
responsive to determining that the target being located in a right upper lobe of the right lung, selecting a first bifurcation of the main carina into the left main bronchus and the right main bronchus, a second bifurcation of the right upper lobar bronchus into an apical segment and a posterior segment, a third bifurcation of the right upper lobar bronchus into the apical segment and a right anterior segment, and a fourth bifurcation of the right upper lobar bronchus into the posterior segment and the right anterior segment;
responsive to determining that the target being located in a middle lobe of the right lung, selecting the first bifurcation, a fifth bifurcation of the right middle lobar bronchus into a lateral segment and a medial segment, a sixth bifurcation of the right lower lobar bronchus into a right anterior basal segment and a right medial basal segment, and a seventh bifurcation of the right lower lobar bronchus into a right lateral basal segment and a right posterior basal segment;
responsive to determining that the target being located in a right lower lobe of the right lung, selecting the fifth bifurcation, the seventh bifurcation, an eighth bifurcation of the intermediate bronchus into the right middle lobar bronchus and the right lower lobar bronchus, and a ninth bifurcation of the right lower lobar bronchus into the right anterior basal segment and the right lateral basal segment;
responsive to determining that the target being located in a left upper lobe of the left lung, selecting the first bifurcation, a tenth bifurcation of the left main bronchus into the left upper lobar bronchus and the left lower lobar bronchus, an eleventh bifurcation of the left upper lobar bronchus into an apicoposterior segment and a left anterior segment, and a twelfth bifurcation of the left lingular bronchus into a superior lingular segment and an inferior lingular segment; and
responsive to determining that the target being located in a left lower lobe of the left lung, selecting the tenth bifurcation, a thirteenth bifurcation of the left lower lobar bronchus into a left medial basal segment and a left posterior basal segment, a fourteenth bifurcation of the left lower lobar bronchus into a left anterior basal segment and the left medial basal segment, and a fifteenth bifurcation of the left lower lobar bronchus into a left lateral basal segment and the left posterior basal segment;
extracting a plurality of virtual images of landmarks in the landmarks subset from the virtual model by deriving an image of each of the landmarks in the landmarks subset from the virtual model;
acquiring a plurality of live images via imaging each of the landmarks in the landmarks subset by the bronchoscope;
acquiring a plurality of live positions by extracting positions of at least three landmarks in the landmarks subset from the plurality of live images, acquiring the plurality of live positions comprising:
moving a tip of the of the bronchoscope to a position of a landmark in the landmarks subset by using a corresponding live image of the plurality of live images, and
extracting the tip's position by using the tracking instrument;
acquiring a plurality of virtual positions by extracting positions of the three landmarks in the landmarks subset from the plurality of virtual images; and
updating the registration transform by using the plurality of live positions and the plurality of virtual positions.

2. A method for navigating an imaging instrument in a branched structure comprising a plurality of landmarks, the method comprising:
selecting a target in the branched structure;
selecting a first landmark from the plurality of landmarks, the first landmark associated with the target;
acquiring a virtual model of the branched structure;
extracting a first virtual image of the first landmark from the virtual model;
acquiring a first live image of the first landmark by the imaging instrument; and
registering the first live image with the first virtual image.

3. The method of claim 2, further comprising:
selecting a second landmark from the plurality of landmarks after acquiring the first live image, the second landmark associated with the target;
extracting a second virtual image of the second landmark from the virtual model;
acquiring a second live image of the second landmark by the imaging instrument;
replacing the first landmark with the second landmark before registering the first live image with the first virtual image;
replacing the first virtual image with the second virtual image before registering the first live image with the first virtual image; and
replacing the first live image with the second live image before registering the first live image with the first virtual image.

4. The method of claim 2, wherein registering the first live image with the first virtual image comprises calculating a registration transform by using a live position of the first landmark acquired from the first live image, a virtual position of the first landmark acquired from the first virtual image, and camera specifications of the imaging instrument.

5. The method of claim 2, further comprising:
selecting a landmarks subset from the plurality of landmarks in a neighborhood of the target, the landmarks subset associated with the first landmark;
extracting a first plurality of virtual images from the virtual model by extracting an image of each of the landmarks in the landmarks subset from the virtual model;
acquiring a plurality of live images via imaging each of the landmarks in the landmarks subset by the imaging instrument; and
registering the plurality of live images with the plurality of virtual images.

6. The method of claim 5, wherein selecting the landmarks subset comprises:
extracting a second plurality of virtual images from the virtual model by extracting an image of each of the plurality of landmarks in the neighborhood of the target from the virtual model;
acquiring a first plurality of virtual positions by extracting positions of each of the plurality of landmarks from the second plurality of virtual images;
acquiring a target virtual position by extracting a position of the target from a target virtual image extracted from the virtual model, the target virtual image including an image of the target;
extracting a virtual positions subset from the first plurality of virtual positions such that a geometric center of the virtual positions subset coincides with the target virtual position; and
selecting a landmark in the landmarks subset from the plurality of landmarks, the position of the landmark in the virtual image of the landmark being included in the virtual positions subset.

7. The method of claim 5, wherein registering the plurality of live images with the first plurality of virtual images comprises:
acquiring a plurality of live positions by extracting positions of at least three landmarks in the landmarks subset from the plurality of live images;
acquiring a second plurality of virtual positions by extracting positions of the three landmarks in the landmarks subset from the first plurality of virtual images; and
calculating a registration transform by using the plurality of live positions and the second plurality of virtual positions.

8. The method of claim 7, wherein navigating the imaging instrument in the branched structure comprises navigating a bronchoscope associated with a tracking instrument in a bronchial tree associated with a main carina, a right lung, and a left lung, wherein:
the right lung comprising:
a right main bronchus;
an intermediate bronchus;
a right upper lobar bronchus;
a right middle lobar bronchus; and
a right lower lobar bronchus; and
the left lung comprising:
a left main bronchus;
a left upper lobar bronchus;
a left lingular bronchus; and
a left lower lobar bronchus.

9. The method of claim 8, wherein selecting the landmarks subset comprises:
responsive to determining that the target being located in a right upper lobe of the right lung, selecting each landmark in the landmarks subset from inside a first region, the first region comprising the right upper lobe and a middle lobe of the right lung;
responsive to determining that the target being located in a middle lobe of the right lung, selecting each landmark in the landmarks subset from inside a second region, the second region comprising the right upper lobe and the middle lobe;
responsive to determining that the target being located in a right lower lobe of the right lung, selecting each landmark in the landmarks subset from inside a third region, the third region comprising the right lower lobe;
responsive to determining that the target being located in a left upper lobe of the left lung, selecting each landmark in the landmarks subset from inside a fourth region, the fourth region comprising the left upper lobe; and
responsive to determining that the target being located in a left lower lobe of the left lung, selecting each landmark in the landmarks subset from inside a fifth region, the fifth region comprising the left lower lobe.

10. The method of claim 9,
wherein responsive to determining that the target being located in the right upper lobe, selecting the landmarks subset comprises:
selecting a first bifurcation of the main carina into the left main bronchus and the right main bronchus,
selecting a second bifurcation of the right upper lobar bronchus into an apical segment and a posterior segment,
selecting a third bifurcation of the right upper lobar bronchus into the apical segment and a right anterior segment, and
selecting a fourth bifurcation of the right upper lobar bronchus into the posterior segment and the right anterior segment;
wherein responsive to determining that the target being located in the middle lobe, selecting the landmarks subset comprises:
selecting the first bifurcation,
selecting a fifth bifurcation of the right middle lobar bronchus into a lateral segment and a medial segment,
selecting a sixth bifurcation of the right lower lobar bronchus into a right anterior basal segment and a right medial basal segment, and
selecting a seventh bifurcation of the right lower lobar bronchus into a right lateral basal segment and a right posterior basal segment;
wherein responsive to determining that the target being located in the right lower lobe, selecting the landmarks subset comprises:
selecting the fifth bifurcation,
selecting the seventh bifurcation,
selecting an eighth bifurcation of the intermediate bronchus into the right middle lobar bronchus and the right lower lobar bronchus, and
selecting a ninth bifurcation of the right lower lobar bronchus into the right anterior basal segment and the right lateral basal segment;
wherein responsive to determining that the target being located in the left upper lobe, selecting the landmarks subset comprises:
selecting the first bifurcation,
selecting a tenth bifurcation of the left main bronchus into the left upper lobar bronchus and the left lower lobar bronchus,
selecting an eleventh bifurcation of the left upper lobar bronchus into an apicoposterior segment and a left anterior segment, and
selecting a twelfth bifurcation of the left lingular bronchus into a superior lingular segment and an inferior lingular segment; and
wherein responsive to determining that the target being located in the left lower lobe, selecting the landmarks subset comprises:
selecting the tenth bifurcation,
selecting a thirteenth bifurcation of the left lower lobar bronchus into a left medial basal segment and a left posterior basal segment,
selecting a fourteenth bifurcation of the left lower lobar bronchus into a left anterior basal segment and the left medial basal segment, and
selecting a fifteenth bifurcation of the left lower lobar bronchus into a left lateral basal segment and the left posterior basal segment.

11. The method of claim 8, wherein acquiring the plurality of live positions comprises:
moving a tip of the of the bronchoscope to a position of a landmark in the landmarks subset by using a corresponding live image of the plurality of live images; and
extracting the tip position by using the tracking instrument.

12. The method of claim 8, wherein acquiring the virtual model comprises reconstructing a three-dimensional model of the bronchial tree from a plurality of computed tomography (CT) images of the bronchial tree.
